(19) United States
(12) Patent Application Publication
(10) Pub. No.: US 2011/0275947 A1
(43) Pub. Date: Nov. 10, 2011

(54) CARDIOVASCULAR POWER SOURCE FOR AUTOMATIC IMPLANTABLE CARDIOVERTER DEFIBRILLATORS

(75) Inventors: Marc D. Feldman, San Antonio, TX (US); Shaochen Chen, San Diego, CA (US); Li-Hsin Han, Sunnyvale, CA (US); Carlos A. Aguilar, Edinburg, TX (US); Arturo A. Ayon, San Antonio, TX (US); C. Mauli A Grawal, San Antonio, TX (US); David M. Lighthart, Portland, OR (US); Devang N. Patel, San Antonio, TX (US); Steven R. Bailey, San Antonio, TX (US); Brian A. Korgel, Round Rock, TX (US); Doh C. Lee, Yuseong-gu (KR); Tushar Sharma, Austin, TX (US); Christopher J. Ellison, Austin, TX (US); Xiaojing Zhang, Austin, TX (US)

(73) Assignee: Board of Regents, The University of Texas System, Austin, TX (US)

(21) Appl. No.: 13/041,932

(22) Filed: Mar. 7, 2011

Related U.S. Application Data
(63) Continuation-in-part of application No. 12/522,012, filed on Jan. 22, 2010.
(60) Provisional application No. 60/883,497, filed on Jan. 4, 2007.
(60) Provisional application No. 60/980,942, filed on Oct. 18, 2007.

(51) Int. Cl. A61N 1/378; H01L 41/113; A61N 1/39; A61B 5/02; B82Y 30/00

(52) U.S. Cl. 600/508; 607/35; 607/5; 607/4; 310/319; 977/762; 977/925

(57) ABSTRACT

Aspects according to the present invention provide a method and implant suitable for implantation inside a human body that includes a power consuming means responsive to a physiological requirement of the human body, a power source and a power storage device. The power source comprises a piezoelectric assembly that is configured to generate an electrical current when flexed by the tissue of the body and communicate the generated current to the power storage device, which is electrically coupled to the power source and to the power consuming means.

### CROSS-REFERENCE TO RELATED APPLICATIONS

This application is a continuation-in-part of U.S. application Ser. No. 12/522,012, which is a 35 U.S.C. §371 national phase application of international application PCT/US2008/000114, filed Jan. 4, 2008, which claims priority from U.S. provisional application No. 60/883,497, filed Jan. 4, 2007 and U.S. provisional application No. 60/980,942, filed Oct. 18, 2007, the entire disclosures of which are all herein incorporated by reference in their entireties.

### FIELD OF THE INVENTION

The present invention generally pertains to a power source whose energy is derived from changes in shape responsive to autonomic movements of the human body. More particularly, to a self-contained power source configured to the implanted in a living organism, such as within a human's heart, such that movement of the heart acting on the power source will cause generation of electrical power.

### BACKGROUND OF THE INVENTION

Generally, patients with reduced systolic function (LVEF<30%) are now recommended to receive an Automatic Implantable Cardiac Defibrillator (AICD). An AICD is a device that is implanted in the chest to constantly monitor and, if necessary, correct episodes of an abnormal heart rhythm. The primary corrective functions of an AICD are to control tachycardia through cardioversion (low-energy shocks to convert the heart rhythm to a more normal rate) and manage fibrillation through defibrillation. Most AICDs are combined with a Bi-Ventricular Pacemaker (BVP), a type of implantable pacemaker designed to simultaneously treat both ventricles when they do not pump in unison. Conventional pacemakers regulate the right atrium and right ventricle (AV synchrony), while BVPs add a third lead to help the left ventricle contract at the same time. Patients with a widened QRS and Stage 3 or 4 congestive heart failure have improved outcome when receiving BVPs. Conventionally, QRS duration is the measured duration of electrical activation of the heart's two main pumping chambers. Recent studies have made it clear that the majority of patients with cardiomyopathy of any cause will benefit from placement of AICD and BVP to both reduce hospital admissions and prolong life.

In 2004, AICDs were implanted in over 100,000 individuals. The rate of replacement of pacemakers and AICDs is dependant on the battery capacity and the degree of pacing and/or occurrence of defibrillation. In medical devices that are implanted, for example, the battery that powers the device such as the AICD must be implanted along with the AICD or be connected to it by leads that pass though the body. The latter option allows the battery to be readily recharged or replaced. However, this option also increases the risk of infection and other complications.

It is estimated that the average life of an internally implanted battery powered AICD is less than half of the normal life span of a patient after having an AICD implanted. Approximately 70% of AICDs and BVPs implanted in 2004 will require replacement because of battery depletion over the next five years. While the longevity of the average AICD patient has increased to 10 years after implantation, only 5% of implants functioned for seven years, and this mismatch poses a significant and ever growing clinical and economic burden. Approximately 90% of AICD failures were caused by normal battery depletion and the shift to dual-chamber models has significantly shortened battery life even further. Moreover, there are now efforts to “piggyback” devices on AICDs and BVPs for additional functionality such as pressure and volume sensors to warn of impending congestive heart failure (CHF), lung impedance sensors to warn of CHF and chemical sensors to provide telemetric measures of glucose, potassium, bun and creatinine, all of which would require additional power.

Therefore, if the battery is implanted, it must someday be replaced and the battery's limited life is a primary failure mechanism in conventional pacemaker and AICD designs. Every time a surgery is performed there is an inherent risk and discomfort to the patient. This, in combination with complications due to bleeding and infection and potential damage to the leads (requiring the leads to also be replaced) during the removal and implantation of a new pacemaker and AICD, make it beneficial for a pacemaker and defibrillator to be implanted that has a life expectancy equivalent to or that exceeds that of the patient. Even the replacement of the battery is a surgical procedure with inherent risks of its own.

One solution to increase the lifetime of such a pacemaker/defibrillator device is to place an electricity/power generator where considerable energy is already available, namely the heart itself. Previous studies have used the body to harvest energy parasitically, that is through mechanisms that capture and make use of energy that is normally dissipated. An excellent example is the surgically implanted piezoelectric polymer that converts mechanical work done by an animal's breathing into electrical power. Another example of parasitic power harvesting from the body was accomplished by placing piezoelectric patches in the heels and soles of soldier's boots to harvest energy from ambulatory motion.

In one exemplary aspect, the present invention can harvests the complex kinetic motion of the heart to provide auxiliary power for, for example, an AICD and/or a BVP. The cardiovascular system as a power source generator is appealing due to its ability to continuously deliver mechanical energy as long as the patient is alive. An AICD that derives its energy from the continuous motion of the heart has a longer lifetime, doesn't have to be replaced as often, can reduce surgeries and the inherent risks that are posed by complications due to bleeding and infection to the leads of the AICD or pacemaker. Conventionally, an AICD detects the onset of tachycardia and attempts to return the heart beat to normal rhythm through pacing and, if pacing is not sufficient to control the tachycardia condition, the defibrillator provides a high-energy shock to stop fibrillation. The battery of the device must supply continuous low (background) current to the device to power the monitoring circuitry, and rapidly delivery high current pulses on demand.

In an additional aspect, the present invention can harvest at least a portion of the kinetic motion of the human body to be used to power any desired power consumption device such as, for example and not meant to be limiting, pressure and volume sensors, chemical sensors, left and right ventricular devices, artificial hearts, and the like. It will be appreciated that the power source of the present invention can be used to provide electrical power to any implanted device that uses electrical power. It is further contemplated that the power source of the present invention could also be used externally of the human body to harvest energy from kinetic motion of bodies, such as for example, water.

Heretofore various methods have been employed for generating electrical energy for electronic implants. In the Snaper et al. U.S. Pat. No. 5,431,694, a piezoelectric generator in the form of a flexible sheet of poled polyvinylidene fluoride that is connected to the skeletal number. The generator is configured to flex with negligible elongation of its surface and can be operably coupled to a power storage device. In the Schroeppel U.S. Pat. No. 4,690,143, a pacing lead is disclosed that has a piezoelectric device in a distal end of the pacing lead. The piezoelectric device is configured to generate electrical energy in response to movement of the implanted pacing lead.

In the Ko U.S. Pat. No. 3,456,134 there is disclosed an encapsulated cantilevered beam composed of a piezoelectric crystal mounted in a metal, glass or plastic container and arranged such that the cantilevered beam will swing in response to movement. The cantilevered beam is further designed to resonate at a suitable frequency and thereby generate electrical voltage.

In the Dahl U.S. Pat. No. 4,140,132 there is disclosed a piezoelectric crystal mounted in cantilevered fashion within an artificial pacemaker can or case, having a weight on one end, and arranged to vibrate to generate pulses which are a function of physical activity. In the McLean U.S. Pat. No. 3,563,245 there is disclosed a pressure actuated electrical energy generating unit. A pressurized gas containing bulb is inserted into the heart whereby the contractions of the heart exert pressure on the bulb and cause the pressure within the bulb to operate a bellows remotely positioned with respect to the heart. This bellows in turn operates an electrical-mechanical transducer.

Further it has been proposed in the Frasier U.S. Pat. No. 3,421,512 to provide a pacer with a biological power supply which generates electrical power for the pacer utilizing a body fluid as an electrolyte. It has also been suggested in the Enger U.S. Pat. No. 3,659,615 to use a piezoelectric bimorph encapsulated and implanted adjacent to the left ventricle of the heart and arranged to flex in reaction to muscular movement to generate electrical power.

Therefore, what is needed is a system and method of using the human body's movement, such as, for example the heart's mechanical contraction/expansion, to deform a power source/generator, such deformation producing an internal dipole moment and creates a voltage. The described power source/generator being configured to overcome many of the challenges found in the art, some of which are described above.

### SUMMARY

In various aspects, there are three types of electro-mechanical devices that can perform energy conversion and they are electrostatic, electromagnetic and piezoelectric. Of the three types, the power source of the present invention uses a piezoelectric type transducer that makes use of electro-mechanical coupling to covert energy. In one aspect, the energy density achievable with piezoelectric devices is potentially greater than comparable electrostatic or electromagnetic devices. In a further exemplary aspect, the materials forming the power source are configured to convert mechanical energy into electrical energy via strain applied to the materials and, as such, lend themselves to devices that operate by bending or flexing, which in the exemplary case of recharging an AICD battery from the human heart is particularly attractive. In one aspect, therefore, the power source of the present invention can use the heart's mechanical contraction/expansion to produce an internal dipole moment and creates a voltage. Of course, it is contemplated that alternative movements of the body, such as exemplarily provided by lung expansion, diaphragm movement, rib bending and the like can provide the desired bending moment on the power source.

In one aspect, the power source of the present invention is configured to generate an electrical current when deformed and is operably coupled to a charge storage device, such as, without limitation, an implanted battery. In a further aspect, the power source of the present invention is adaptable to the attached to a structure, such as, for example and without limitation, a pacing lead that can be repetitively bent, and while bent, to generate an electric current.

Accordingly, aspects according to the present invention provide a method and implant suitable for implantation inside a human body that includes a power consuming means responsive to a physiological requirement of the human body, a power source and a power storage device. The power source comprises a piezoelectric assembly that is configured to generate an electrical current when flexed by the tissue of the body and to communicate the generated current to the power storage or any buffer device, which is electrically coupled to the power source and to the power consuming means. In optional aspects, it is contemplated that the piezoelectric assembly can be configured to not be sheathed or at least a portion of the piezoelectric assembly can be sheathed.

It is contemplated that the power consuming means can comprise, for example and without limitation, the nominal power requirements of the AICD and/or pacemaker, implantable sensing devices, such as for example, right and left volume and pressure sensors, lung impedance sensors to warn of impending heart failure, and chemical sensors to provide telemetric measures of, for example, glucose, potassium, bun and creatinine Potential “piggyback” device increase the power demands on the implanted power source.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention.

### DETAILED DESCRIPTION OF THE DRAWINGS

The accompanying drawings, which are incorporated in and constitute a part of this specification, illustrate certain aspects of the instant invention and together with the description, serve to explain, without limitation, the principles of the invention and like reference characters used therein indicate like parts throughout the several drawings:

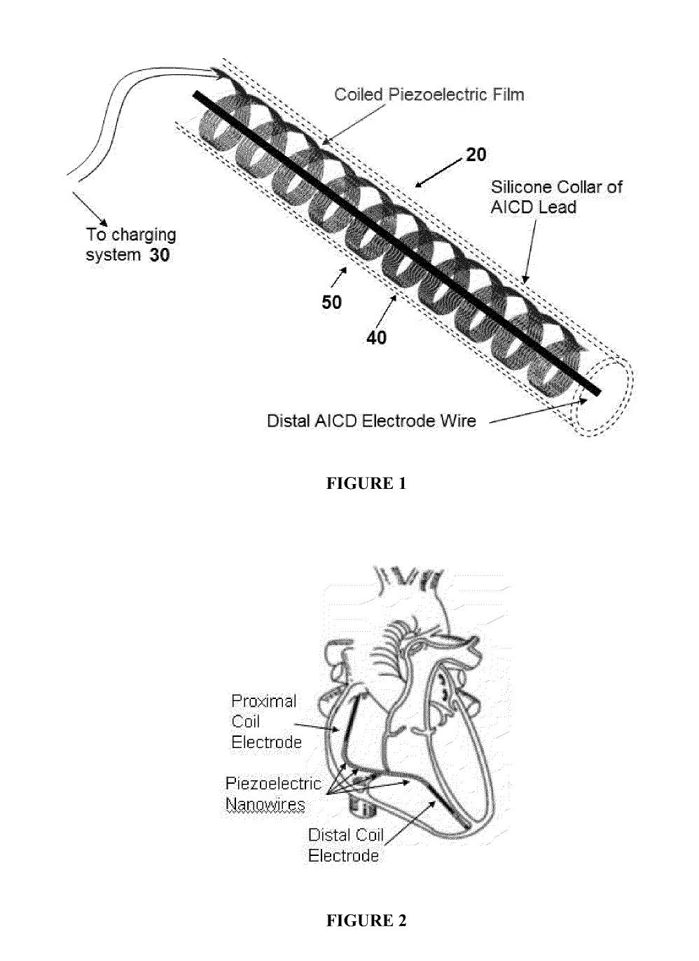

FIG. 1 is a partial perspective view of one embodiment of an exemplary power source of the present invention mounted therein a portion of an AICD lead.

FIG. 2 is a partial cutaway view of a power source of the present invention embedded therein the heart of a subject, showing the power source spaced from the proximal and distal coil electrodes of the pacing lead.

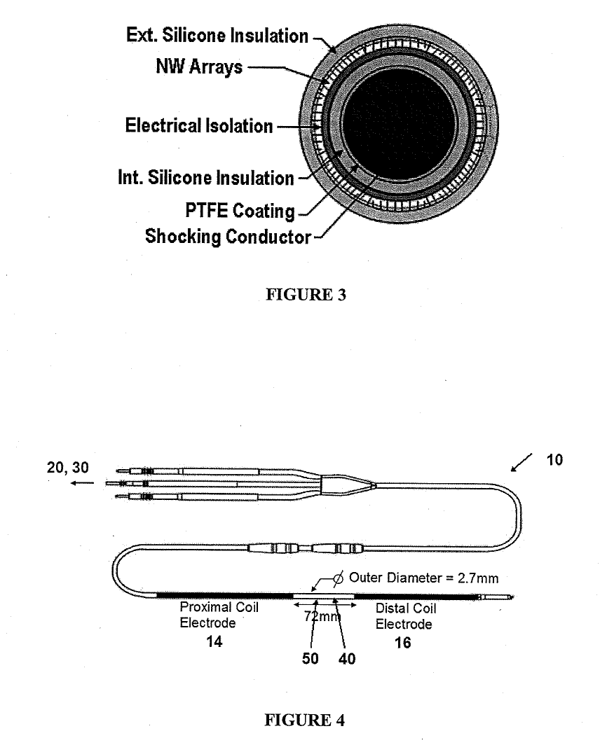

FIG. 3 is a cross-sectional view of a second embodiment of an exemplary power source of the present invention, showing a piezoelectric assembly surrounding the shocking conductor of a pacing lead.

FIG. 4 is a side elevation view of an exemplary pacing lead with the power source of FIG. 4 disposed therein the pacing lead therebetween the proximal and distal coil electrodes of the pacing lead.

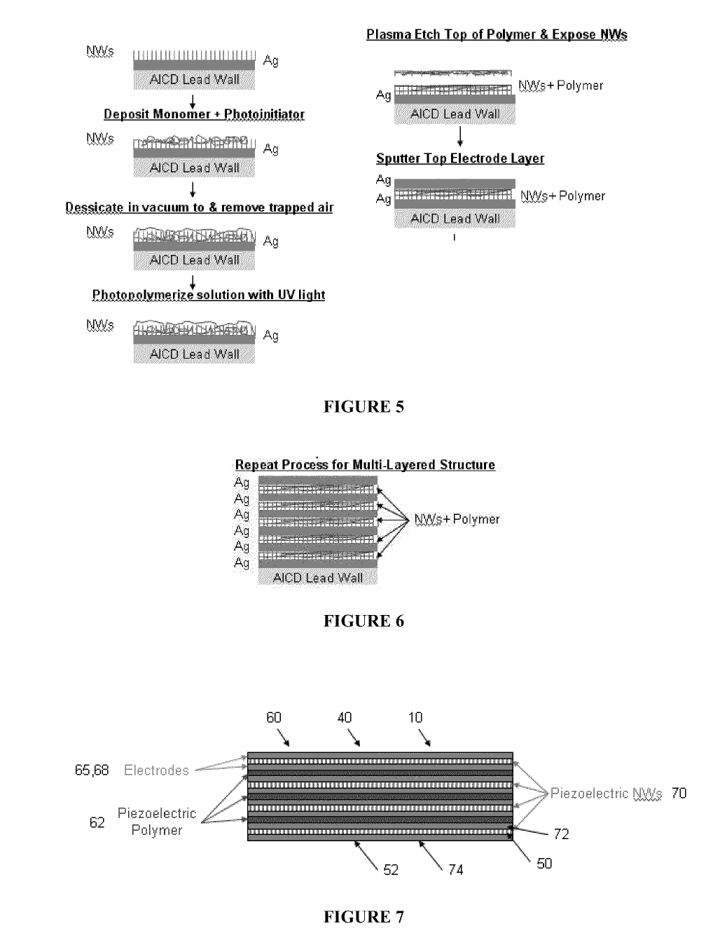

FIG. 5 is a schematic illustration of an exemplary build up of an exemplary power source, showing a single layer piezoelectric assembly mounted to the exterior AICD wall.

FIG. 6 is a schematic illustration of a multilayer piezoelectric assembly mounted to the exterior AICD lead wall.

FIG. 7 is a schematic illustration of a multilayer piezoelectric assembly.

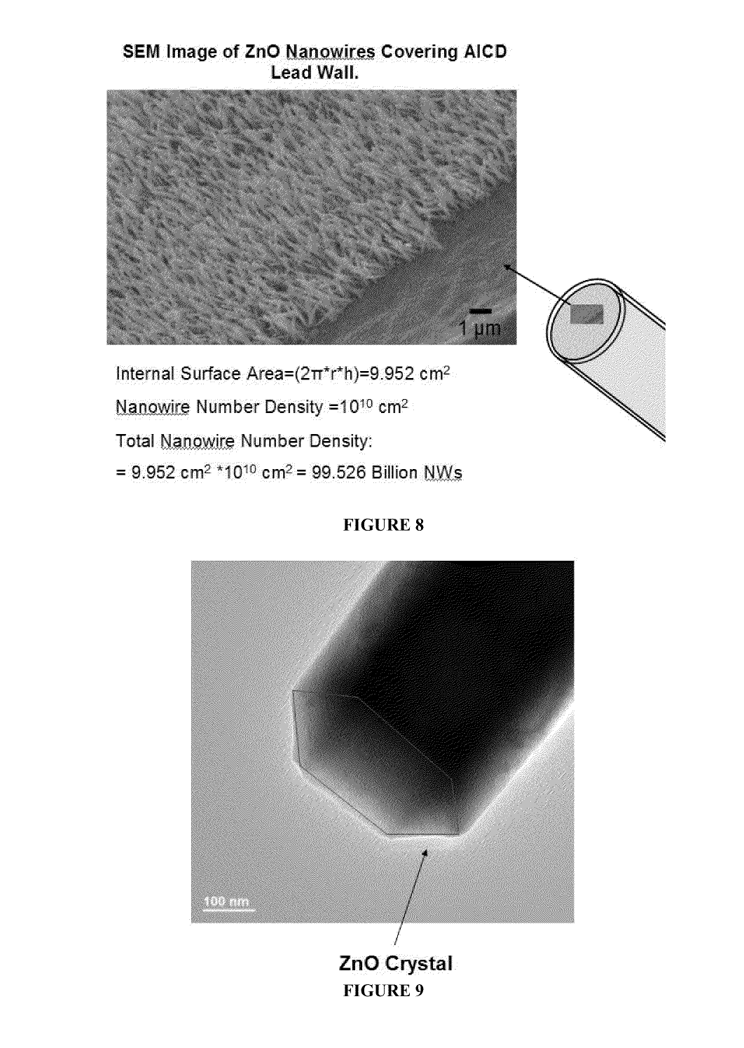

FIG. 8 is a SEM image of exemplary ZnO nanowires extending therefrom an Ag layer that covers the exterior AICD lead wall.

FIG. 9 is a SEM image showing a perspective view of a distal end of a ZnO nanowire, showing its generally hexagonal shape.

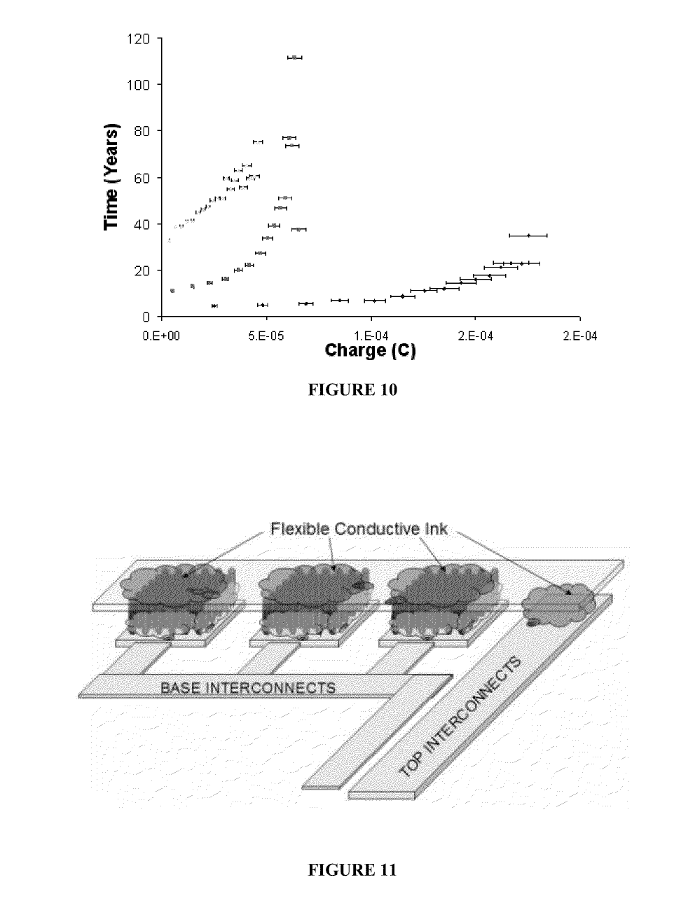

FIG. 10 is a graphical representation of the charge generated by an exemplary power source of the present invention over the course of time.

FIG. 11 is a schematic illustration of a multilayer piezoelectric assembly having flexible conductive ink.

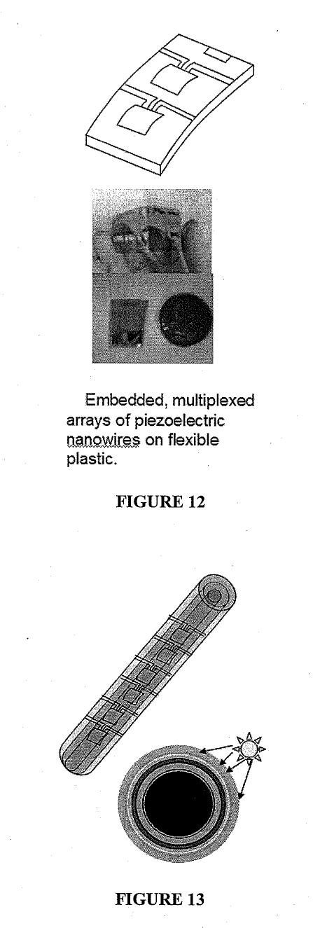

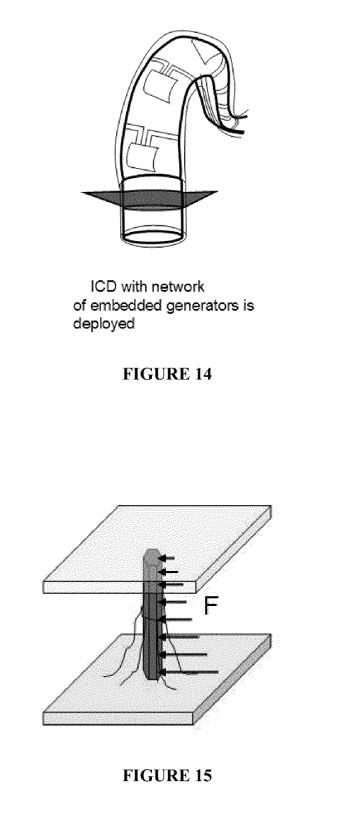

FIGS. 12-14 illustrate an exemplary embodiment showing the fabrication of an ICD lead using base films, such as shown in FIGS. 11 and 12.

FIG. 15 is a schematic illustration showing electrodes that are positioned at both ends of the nanowire.

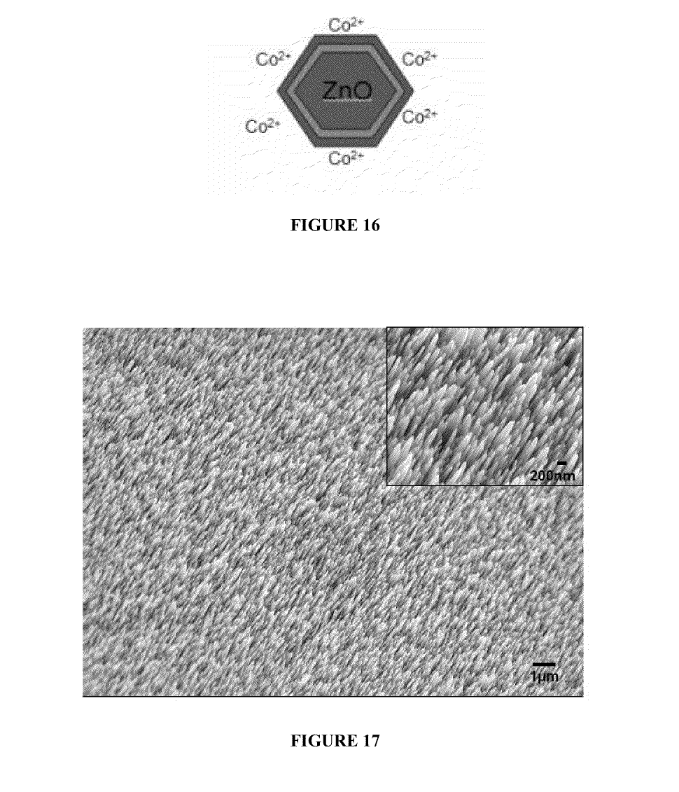

FIG. 16 is a schematic illustration of a doped nanowire. In various examples, the dopants are dispersed into the crystal lattice of the array of nanowires isotropically by, for example and not meant to be limiting, conventional electrochemistry and/or core-shell methodologies.

FIG. 17 is a scanning electron micrograph of as-grown ZnO nanowires on a lithographically patterned Kapton substrate. As shown, the nanowires are highly oriented with there bases well attached to the patterned electrodes. The inset is a magnified view of the nanowires.

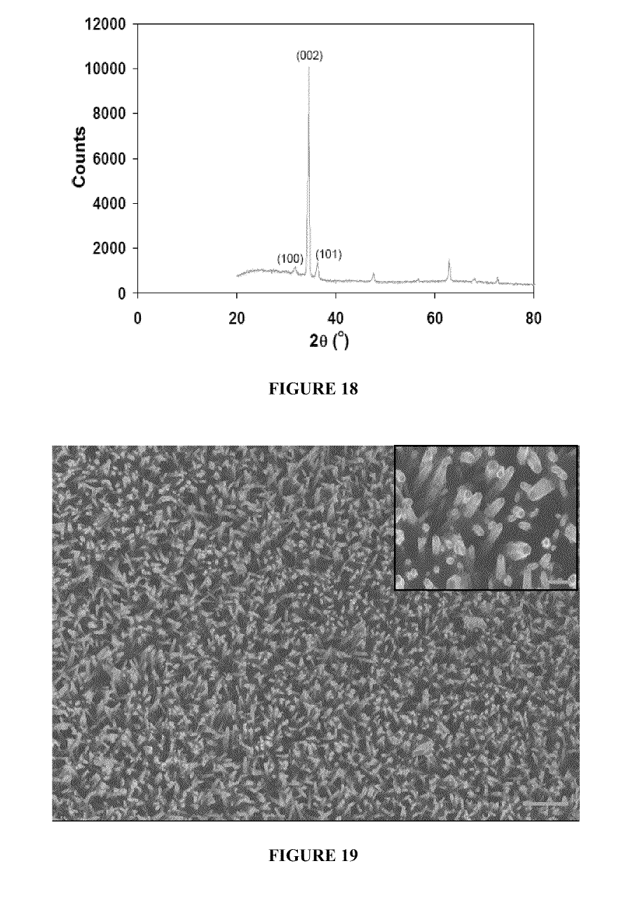

FIG. 18 is a graph showing X-Ray diffraction of the ZnO nanowires on a lithographically patterned Kapton substrate. The graph shows that the nanowires of the array are highly oriented to the base as demonstrated by the massive enhancement of the (002) peak.

FIG. 19 is a scanning electron micrograph of highly oriented ZnO nanowires embedded in PMMA polymer substrate. The inset is a magnified view of the nanowires.

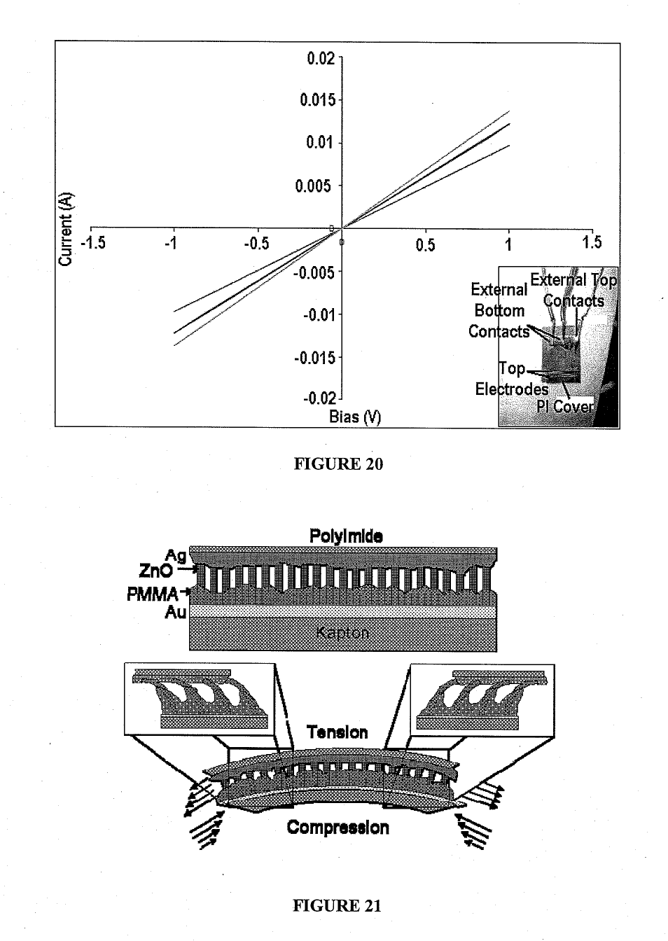

FIG. 20 is a graph showing two-point electrical measurements of exemplary piezoelectric assembly arrays of the present invention after the upper electrode has been cast. The contacts and nanowires are well attached as demonstrated by the linear voltage (I-V) traces. The inset is a photograph of an exemplary piezoelectric assembly.

FIG. 21 is an exemplary schematic of the device before and during systole. As the piezoelectric assembly array is pulled into compression, the polymer surrounding the nanowires is pulled into tension due to the differing radii of curvature. The tensile stress forces the nanowires to bend and create energy through the piezoelectric effect.

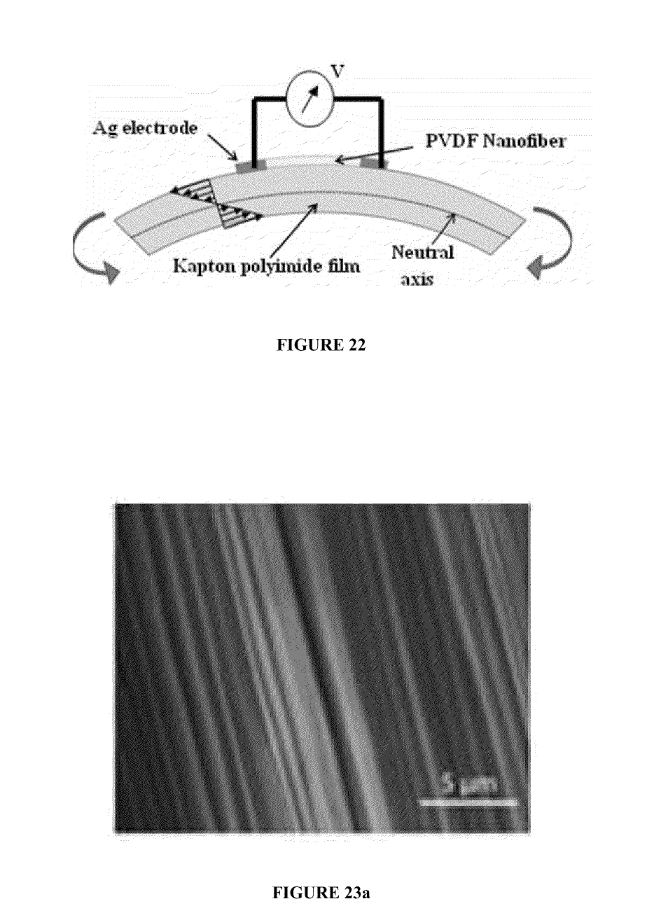

FIG. 22 is a schematic illustration of a single PVDF nanofiber or PVDF nanofilm overlying and bonded to a Kapton polyimide film substrate, showing the two respective ends of the PVDF nanofiber being bonded to the substrate, and showing that imposed mechanical stress induces a piezoelectric field along the PVDF nanofiber.

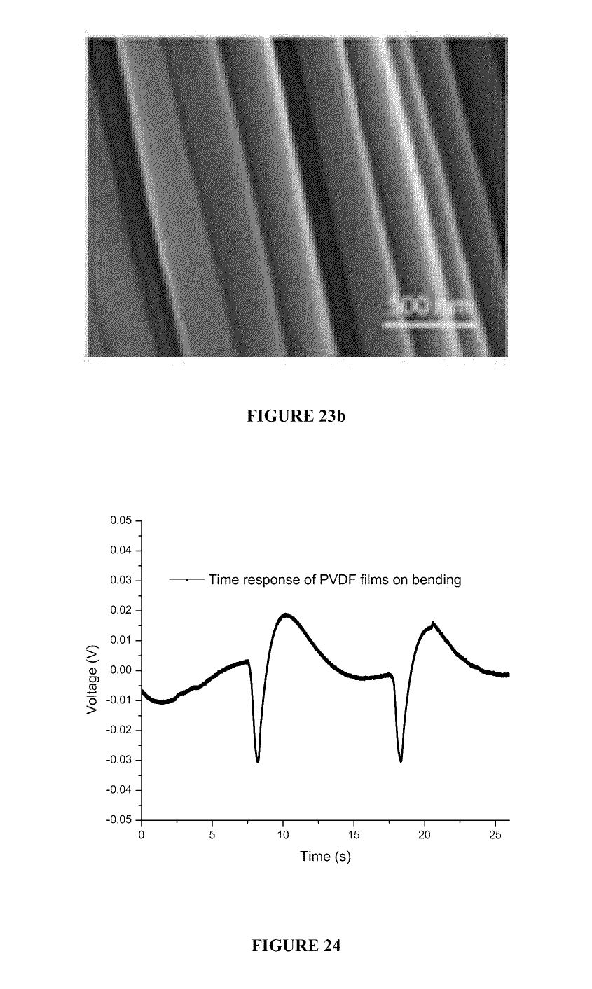

FIG. 23a is a SEM image showing a plurality of substantially parallel PVDF nanofibers. A scale of 5 μm is shown.

FIG. 23b is a SEM image showing a plurality of substantially parallel PVDF nanofibers. A scale of 500 nm is shown.

FIG. 24 is a graph showing the voltage response over time of PVDF films of the present invention upon applied bending.

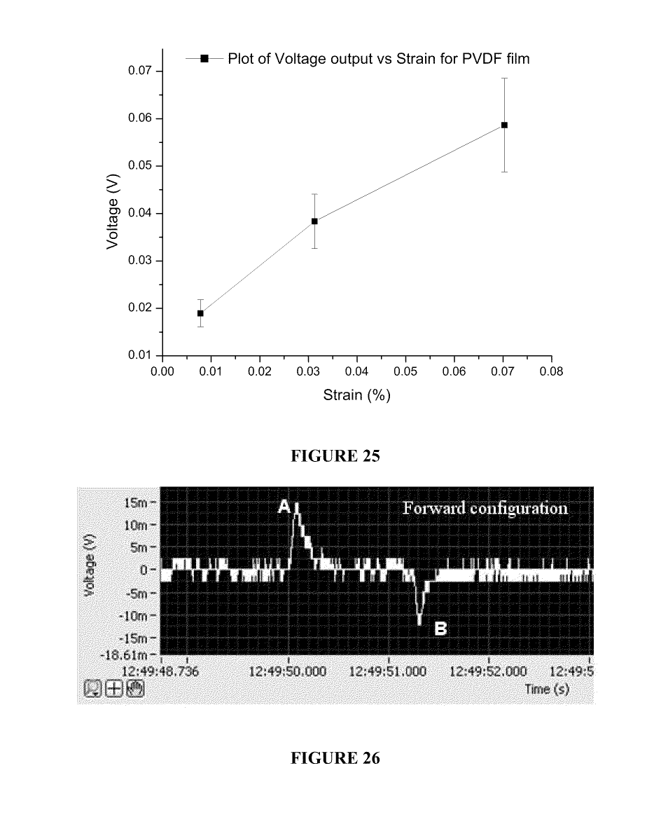

FIG. 25 is a graph showing the voltage response over percentage strain of PVDF films of the present invention upon applied bending.

FIG. 26 is a graph showing the voltage output of the system illustrated in FIG. 22 when the substrate is stretched and released repeatedly when the respective positive and negative probes of the measurement system are connected to the respective positive and negative ends of the PVDF nanofilm.

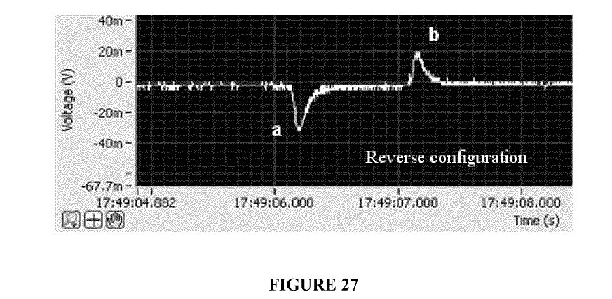

FIG. 27 is a graph showing the voltage output of the system illustrated in FIG. 22 when the substrate is stretched and released repeatedly when the respective positive and negative probes of the measurement system are connected to the respective negative and positive and ends of the PVDF nanofilm.

### DETAILED DESCRIPTION OF THE INVENTION

The present invention may be understood more readily by reference to the following detailed description of the invention and the examples included therein and to the figures and their previous and following description.

Before the present systems, articles, devices, and/or methods are disclosed and described, it is to be understood that this invention is not limited to specific systems, specific devices, or to particular methodology, as such may, of course, vary. It is also to be understood that the terminology used herein is for the purpose of describing particular embodiments only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used in the specification and the appended claims, the singular forms “a,” “an” and “the” include plural referents unless the context clearly dictates otherwise. Thus, for example, reference to “a layer” includes two or more such layers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Embodiments according to the present invention are described below with reference to block diagrams and flowchart illustrations of methods, apparatuses (i.e., systems) according to an embodiment of the invention. Accordingly, blocks of the block diagrams and flowchart illustrations support combinations of means for performing the specified functions and combinations of steps for performing the specified functions.

In one aspect of the present invention, an implant 10 of the present invention can comprise a power consuming means 20, a power storage device 30 and a power source 40, which are operably coupled together. The power consuming means can be a user device, such as, for example and without limitation, a pacemaker, an AICD, a BVP, an insulin pump, right and left ventricular assisted devices, an artificial heart, chemical sensors, pressure and volume sensors, telemetric devices, and the like. In one aspect, the power consuming means can be configured to respond to a physiological requirement of the body. The details of the exemplified user devices are not important to the present invention and are not included herein.

In one exemplary aspect, it is contemplated that the exemplified power source can be used as a sensor for myocardial tensiometry. In this aspect, the contractility of the cardiac muscle can be sensed and a signal indicative of the strength of contraction can be generated. In a further aspect, the contractility signal can be analyzed to provide tensiometric measurements over time. In one example, the derived tensiometry measurements can be used for appropriate applicability of desired inotropic agents.

In various aspects, a piezoelectric structure designed to efficiently convert the kinetic motion of the heart into power for an implantable device should be flexible, nontoxic, possess a high piezoelectric coefficient (mechanical-to-electrical conversion efficiency), not present any load, utilize multiple inputs, sustain a long lifetime, and be able to act synergistically with the implantable device lead. Any technique that harvests the heart's energy is complicated by the requirements that it must be totally unobtrusive and must not increase the load on the heart. The relationships between the response of a piezoelectric element and the force applied depend on three factors: the material's piezoelectric properties, the mechanical or electrical excitation vector, and the structure's dimensions and geometry. Since the dimension of an AICD lead and the excitation vector are generally substantially fixed components, the material properties of a piezoelectric of the present invention are tailored to extract the largest possible response.

Most high-performance bulk piezoelectric materials such as lead-zirconate-titanate (PZT) and lead-magnesium-niobate (PMN) contain at least 60% lead, which is toxic. Although there have been concerted efforts to develop lead-free piezoelectric materials, no effective alternative has to date been identified. Bulk binary systems of orthorhombic perovskite-type (K_{0.5}Na_{0.5})NbO₃ and hexagonal pseudo-ilmenite-type LiTaO₃ have been fabricated with piezoelectric properties near actuator-grade PZT (PZT5H) [1]. Alternatively, thin films of other similar inorganic piezoelectric materials such as, for example, barium titanate (BaTiO₃) and potassium niobate (KNbO₃), with high stiffness and strong piezoelectric activity in bulk poly-crystalline form have also been produced as thin as tens of microns. However, conventional thin films of such materials typically can not be synthesized or sintered onto AICD lead materials without melting or severely compromising the integrity of the plastic lead. Moreover, ceramic structures comprised of such materials cannot be generally be implemented as energy scavenging means into an AICD/BVP lead without heavy contributions to lead stiffness. Furthermore, thin film ceramic structures undergoing cyclic loading are susceptible to cracking and fracture, which would short-circuit the device. Thus, if a lower-temperature synthesis could be employed and precipitation from a solution or vapor could produce a continuous thin film of such displacive ferroelectrics, the films would still suffer from susceptibility to cracking and subsequent electrical short-circuit.

It is contemplated that the power storage device 30 can comprise any device that is capable of storing and dispersing electrical energy. For example, the power storage device can comprise at least one battery, at least one capacitor, and the like. The selection of the appropriate battery, capacitor, and/or rectifier that would be suitable for the implant 10 is well within the skill of one skilled in the art.

In one aspect, the power source 40 of the present invention comprises a piezoelectric assembly 50 that is configured to be sufficiently flexible to be implantable in a tissue of the body that undergoes movement. In one aspect, the piezoelectric assembly is surrounded by a non-porous sheath 52 that allows the piezoelectric assembly to be isolated from the surrounding tissues and fluids when implanted within the body. In another aspect, the piezoelectric assembly is configured to generate an electrical current when flexed by the tissue of the body. As one will appreciate, the piezoelectric assembly is flexible and can be configured to be fixed to a selected anatomical element that undergoes autonomic flexural movement. For example, and without limitation, the anatomical element can include heart muscle, diaphragm muscle, ribs, and the like.

In one preferred embodiment, the power source is embedded therein a portion of an AICD or pacemaker lead which is fixed to the free wall of the right ventricle. In this aspect, because the right ventricle free wall undergoes the most displacement of any portion of the cardiovascular system, the power source will be strained more and therefore produce more charge than if it was implanted in other potential anatomical locations. The power source can be attached to the desired anatomical element by conventional means, such as, sutures, surgical adhesives, staples, and the like. Thus, in one exemplary aspect, an AICD or pacemaker lead that contains the power source can be selectively attached to the desired anatomical element, such as, for example, to the free wall of the ventricle. In this aspect, the lead's construction protects the power source from fluids, macrophages, leukocytes and the like that are present in the body around the anatomical element.

It is contemplated, and as described in more detail below, that the piezoelectric assembly 50 can comprise a combination of PVDF nanowires or films made of one or more layers of PVDF.

In one embodiment, the piezoelectric assembly 50 can comprise a nanocomposite structure 60 that surrounds a substantially flexible substrate. The substrate can exemplary be formed from a polymer, hydrogel, composite, or the like, which can have piezoelectric, conducting, and/or dielectric properties. In a further aspect, the nanocomposite structure can comprise at least one poled sheet or at least one unpoled sheet. Optionally, it is contemplated that the at least one poled or unpoled sheet can comprise in-site poling.

In various aspects, it is contemplated that the nanocomposite structure can comprise a flexible piezoelectric film 62, which can exemplarily be formed from, for example and without limitation, a polyvinylidenefluoride (PVDF) film, a copolymer of polyvinylidene difluoride and trifluoroethylene (PVDF-TrFE) film, and/or composites of PVDF with PZT and PMN. It is contemplated that the piezoelectric film can be fabricated in various desired geometric shapes, sizes and patterns. In one exemplary aspect, each poled sheet can have an upper electrode layer 66 connected to the top surface 64 of the film and a lower electrode layer 68 that is connected to the bottom surface 65 of the film. In another exemplary aspect, successive layers of the flexible piezoelectric film can be built up by bonding the respective layers together by conventional processes such as, for example and without limitation, thermal curing, melting followed by re-crystallization, a commercial adhesive, and the like. One skilled in the art will appreciate that the conformability of the material permits its integration into AICD leads without substantial contributions to lead stiffness. However, PVDF has a relatively low piezoelectric coefficient (<16%). Thus, in order to increase the piezoelectric activity of such a material to a desired level, forming a composite comprising multiple stacked layers is preferred.

In one exemplary aspect, and as explained in more detail below, the selected thickness of the individual respective PVDF layers in the formed built up nanocomposite structures can range between about 1 nanometer to about 10 microns. It is further contemplated that, in a multilayer nanocomposite structure configuration, the PVDF layers would alternate with a second layering component which could be, for example and without limitation, an insulator (polymer or inorganic), a metal, conducting polymer or polymer composite, inorganic nanowire, and the like. In this aspect, the alternating layer structure substantially confines or isolates the spaced PVDF layers between intervening layers that are not formed from PVDF. This nanocomposite structure configuration may enhance the crystal content and piezoelectric properties of the formed piezoelectric assembly 50.

In a further embodiment, the nanocomposite structure can comprise at least one layer of nanowires (NWs) 70 that are operatively coupled to the same upper electrode layer 72 and opposed lower electrode layer 74 as the PVDF film. In this aspect, the strain experienced by an array of piezoelectric NWs is higher than in a similar sized bulk polycrystalline piezoelectric material. Because the total surface to volume ratio of a NW array is higher than a polycrystalline film, the individual NWs are able to deflect more and experience a higher strain and in turn, are able to produce more energy per unit area through the piezoelectric effect. Moreover, single-crystalline materials, such as NWs, generally have larger electro-mechanical coefficients than their bulk polycrystalline counterparts due to the lack of defects. This is because piezoelectric NWs can be synthesized with lower defects and practically no grain boundaries, which can facilitate more mobility in the domain walls and create higher electro-mechanical coupling coefficients. Additionally, NW arrays offer a potentially fail-safe technology because if one or a thousand of the respective individual NWs fracture, the generator will not short circuit and stop producing power as it would in a conventional single film. In the present invention, there are a large number of active inputs (>10¹⁰ per cm²) that would be producing energy, which allows the generator of the present invention to last longer than macroscopic counterparts. In a further aspect, the size reduction of this embodiment of the present invention offers the potential to stack arrays on top of one another for three-dimensional architectures without significantly altering the overall dimensions or stiffness of the energy harvester.

The piezoelectric activity of individual nanowires (NWs) has been studied where the mechanical excitation was induced by deflection of a single ZnO NW from an atomic force microscope (AFM) probe tip and the resulted electric response was sensed through the probe tip. The output of the NW was 10⁻¹⁷ J in one discharge event. The piezoelectric response of a single BaTiO₃ NW has also been studied through a miniaturized flexure stage that applies a periodic tensile load and the generated voltage was drained off into patterned contacts. Since individual nanoelectronic power sources provide only miniscule amounts of work, the actions of billions or more must be harnessed in parallel to result in significant activity. In one aspect of the present invention, the piezoelectric assembly 50 can use a flexible substrate that can be configured to conform to the AICD and BVP lead and move with the mechanical displacement of the RV. In various aspects, the piezoelectric assembly 50 of the present invention can incorporate piezoelectric NWs that have a very high energy density and large flexibility, permitting their integration into conventional AICD and BVP leads; can be configured so the NWs receive adequate strain to produce energy through the piezoelectric effect; and can be configured to not add stiffness to the lead and thus not present any additional load on the heart. Further, the piezoelectric assembly 50 of the present invention allows for the production of ordered arrays of piezoelectric NWs with high densities (>10¹⁰ per cm²) directly on a flexible device and the integration of the piezoelectric without any processing or registry to individual nanowires.

In one aspect, the at least one layer of nanowires is configured to form the outermost layer of the piezoelectric assembly 50 so that the maximum amount of stress when the power source is bent can be directed to the at least one layer of nanowires.

In one aspect, the nanowires can be formed from an array of piezoelectric crystals, such as, for example and without limitation, Polyvinylidene Fluoride (PVDF), Zinc Oxide (ZnO) crystals, Gallium Nitride (GaN) crystals, Lead Zirconate-Lead Titanate (PZT) crystals, lead manganese niobate (PMN) crystals, Barium Titanate (BaTiO₃) crystals, Quartz (SiO₂) crystals, Lithium Niobate (LiNbO₃) and Lithium Tantalate (LiTaO₃) crystals, Potassium Niobate (KNbO₃) and Potassium Niobate-Tantalate (KNbTaO₃) crystals, Cadmium Sulfide (CdS) crystals, Cadmium Selenide (CdSe) crystals, Aluminum Nitride (AlN) and the like. For example, an embodiment of the power source is described herein comprises ZnO crystals. One skilled in the art will appreciate that it is contemplated that the piezoelectric crystal could be comprised of various morphologies beyond nanowires, such as but not limited to “thin” films, microwires, branched networks of nanowires and microwires or coils and comprise any suitable piezoelectric crystal or combinations of piezoelectric crystals. It is also contemplated that the crystals could contain combinations of two different crystal structures for a binary system or heterostructure such as, for example and without limitation, (KNa)NbO₃—LiTaO₃ or ternary systems, such as, for example and without limitation, (KNa)NbO₃—LiTaO₃—LiSbO₃.

In one aspect, it is contemplated that the contemplated use of PVDF nanowires or films advantageously allows for the construction of a nanocomposite structure that is both biocompatible and non-dissolvable therein mammalian subjects. This allows for the PVDF nanowires to be confidently used for extended implanted use in either sheathed, partially sheathed, or non-sheathed configurations.

The nanowires act to increase the capacitance or energy density of the multi-layer structure and its ability to generate charge. In a further aspect, the layer of nanowires can be encapsulated in a polymeric matrix, such as, for example and without limitation, a polyethylene material, a polyurethane material, a poly(methylmethacrylate), a polyimide (PI, Kapton), a polyamide (PA, Nylon), a polyethylene terephthalate (PET, Mylar, Dacron), a polypropylene, polytetrafluoroethylene (PTFE, Teflon) and the like. Embedding the nanowires in the polymeric matrix acts to transfer the mechanical load into the length of the nanowires and to add mechanical stability to the nanowire array.

Optionally, it is contemplated that the layer of nanowires can be coupled to an underlying substrate. As shown in FIG. 22, the PVDF nanowires can be operatively coupled to an underlying surface of a substrate, which is exemplarily, and not meant to be limiting, shown as a Kapton polyimide film. In one aspect, it is contemplated that the layer of nanowires can comprise a plurality of substantially aligned PVDF nanofibers. As shown in FIGS. 23a and 23b, it is further contemplated that the layer of nanowires can comprise a plurality of PVDF nanofibers can be positioned thereon the substrate such that the respective PVDF nanofibers are positioned substantially parallel to each other. Optionally, it is contemplated that the PVDF nanowires can be coupled to the underlying substrate in any orientation relative to the substrate, for example and not meant to be limiting, the PVDF nanowires can be wrapped or otherwise coiled about the underlying substrate. PVDF nanowires formed from PVDF nanofibers possess high flexibility, which provides minimum resistance to external mechanical movements such as heart motion.

It is further contemplated that the polymeric matrix can comprise composites of crystal piezoelectrics and piezoelectric polymers with conventional polymers. For example, and without limitation, the polymeric matrix can comprise polyvinylidene difluoride (PVDF) film, a copolymer of polyvinylidene difluoride and trifluoroethylene (PVDF-TrFE), a composite material of lead zirconate-lead titanate (PZT) and polyvinylidene difluoride (PVDF), a composite material of lead zirconate-lead titanate (PZT) and rubber, a composite material of PVDF and rubber, and the like.

It be appreciated that the respective electrodes of respective layers of the bimorph structure are conventionally coupled to the power storage device. In a further aspect, the coupled electrodes to the piezoelectric crystals could be comprised of conducting or semiconducting nano or micro-wires and/or -particles, thin films, and conducting polymers. In another aspect, it is contemplated that the electrodes, substrate or piezoelectric film can be selectively patterned to maximize the electromechanical coupling and transfer of charge from the device to the electrodes. In this aspect, the gap between the electrodes can be filled with another non-conducting, semiconducting or conducting nano or micro-wires and/or -particles, thin films and/or conducting polymers. It is also contemplated that the surfaces of the electrodes may be treated with molecular surface coatings with terminal end groups such as but not limited to (CH₃, F) to tune the contact resistance that develops between the piezoelectric crystals and neighboring contacts. Optionally, in order to lower the impedance of the piezoelectric assembly 50, all the electrodes can be connected in parallel by switching polarities between electrodes on opposite film/layer surfaces to avoid charge cancellation.

In operation, when the structure is bent by the movement of the anatomical element, the layer (or layers) of nanowires are pulled into tension by the surrounding polymeric matrix and negatively strained or contracted in the direction of the neighboring electrodes. The opposing bottom surface(s) are pushed into compression as a result of the differing radii of curvature. The load applied acts to produce a voltage difference across the respective upper and lower electrodes of each individual layer through the dominant “3-3” longitudinal mode of piezoelectric coupling in the piezoelectric film. Restoring the power source to its original shape acts to discharge the induced charge into an exemplary conditioning circuit.

In various aspects, the signal discharged by the power source can be full-wave rectified through a diode bridge and subsequently filtered into capacitors, such as exemplary solid-state capacitors, which can act to store the charge. In another aspect, the capacitors can be configured to discharge and charge the battery when the voltage on the capacitors has built up to a degree sufficient to overcome the voltage supplied by the battery. Of course, it is contemplated that the process of charging and discharging the capacitors in continuously repeated, which thereby increases the lifetime of the user device. The multilayer bimorph structure described above can advantageously significantly reduce the required time to charge a user device such as an ACID.

In one preferred aspect, and as shown in FIG. 3, the power source can be embedded therein a portion of an ACID or pacemaker lead. Conventionally, such a lead 12 comprises a proximal electrode 14 and a distal electrode 16 that are configured to be couple to the ACID or pacemaker power supply. It is contemplated that the proximal and distal electrodes can be coil electrodes. In one aspect, the power source can be encapsulated within an intermediate portion of the pacing lead between the respective proximal and distal electrodes. Further, the power source is configured to be electrically isolated from the external environment and also from any internal conductors which may be placed within the lumen of the catheter/lead body.

In a further aspect, and as shown in FIG. 1, the piezoelectric assembly 50 of the power source can be configured into a spiral coil and mounted therein a portion of the ACID or pacemaker lead. Preferably, the spiral coil is mounted therein the intermediate portion of the pacing lead between the respective proximal and distal electrodes.

In still a further embodiment of the present invention and referring now to FIGS. 3-6, the piezoelectric assembly 50 of the power source can comprise a single nanowire layer that comprises an array of oriented nanowires that are operatively coupled to an upper electrode layer and an opposed lower electrode layer. As noted above, the nanowires can exemplarily be formed from an array of piezoelectric crystals that are embedded in a polymeric material. Also as noted above, for example and without limitation, the electrode layers can also be formed from semiconducting or conducting nano and microwires, flexible conducting polymers, and the like.

In one aspect, and referring to FIG. 5, a schematic methodology for forming a single nanowire layer is illustrated. Here a lower electrode is deposited on the outermost wall or sheath. The array of ZnO nanowires are grown and oriented thereon the exposed surface of the lower electrode. A polymeric material is then deposited on the grown crystals to encapsulate the array of nanowires. In one preferred step, the polymeric material comprises methylmethacrylate and a photoinitiator. A vacuum can be applied to desiccate the deposited materials and to remove any trapped air. Subsequently, the deposited materials can be photo polymerized via application of a conventional UV light.

In one aspect, generally all of the nanowires of the array of oriented nanowires extend upwardly away from the lower electrode and are generally oriented parallel to a common array axis that is positioned relative to the surface of the lower electrode. It will be appreciated however, that it is contemplated that some of the nanowires of the array of nanowires will not extend substantially parallel to the common array axis. In a further aspect, it is contemplated that the common array axis could be at any desired angle relative to the surface of the lower electrode, for example, the common array axis could be positioned between about 70° to 110° with respect to the surface of the lower electrode, and preferably is positioned about 90° or normal to the surface of the lower electrode.

Next, the top portion of the built up composite structure can be reduced to expose the distal ends of the array of nanowires. This reduction can be accomplished using a plasma etcher. Finally, an upper electrode layer can be applied to the exposed surface of the built up composite structure. In one exemplary aspect, the respective upper and lower electrode can be formed from, without limitation, gold, indium tin oxide (InSn0₂), silver, aluminum, flexible conducting epoxy, and the like. One skilled in the art would appreciate that the upper and lower electrodes are coupled as outlined above to the power storage device.

In another example, the conducting epoxy used, for example 101-42, Creative Materials Inc., as the upper electrode can provide excellent adhesion to metal-oxide surfaces and be very resistant to flexing and creasing. The thin bottom Au contact however can degrade from cyclic strains over time. To reduce the effect of the strain, the planar contacts can be formed into periodic wave-like geometries that can be stretched or compressed to large levels of strain without loss of performance. These structures accommodate large compressive and tensile strains through changes in the wave amplitudes and wavelengths rather than through destructive strains in the materials themselves. The wave-like geometry as the base electrode may lessen the degradation of the contact over time, facilitating a longer device lifetime.

Referring to FIG. 7, it is contemplated that the process could be repeated as necessary to build a piezoelectric assembly that has a plurality of nanowire layers. In this aspect, the plurality of nanowires can be positioned in stacked relationship relative to each other.

It is further contemplated that, as disclosed in the structure outlined above, that the piezoelectric assembly can further comprises at least one poled sheet of flexible piezoelectric film. It will also be appreciated that it is contemplate that the piezoelectric assembly can comprise a plurality of layers that comprise at least one nanowire layer and at least one poled sheet of flexible piezoelectric film. Optionally, the respective nanowire layers and the respective sheets of flexible sheets can be stacked in any desired orientation.

It is contemplated that the piezoelectric film can comprise conventional polyvinylidenefluoride film as well as Cs of materials such as, for example and without limitation, Zinc Oxide (ZnO) thin film, Gallium Nitride (GaN) thin film, Lead Zirconate-Lead Titanate (PZT) thin film, Barium Titanate (BaTiO₃) thin film, (Pb,Sm)TiO3 thin film, Lithium Tantalate (LiTaO₃) thin film, Lithium Niobate (LiNbO₃) thin film, Lead Manganese Niobate (PMN) thin film, Potassium Niobate (KNbO₃) and Potassium Niobate-Tantalate (KNbTaO₃) thin film, Quartz (SiO₂) thin film, Cadmium Sulfide (CdS) thin film, Cadmium Selenide (CdSe) thin film, Aluminum Nitride (AlN) thin film and the like.

As shown in FIG. 4, the exemplified piezoelectric assembly can be positioned therein a portion of the pacing lead of a conventional ACID. As shown, in one aspect, the power source can be encapsulated within an intermediate portion of the pacing lead between the respective proximal and distal electrodes. Further, the power source is configured to be electrically isolated from the external environment and also from any internal conductors which may be placed within the lumen of the catheter/lead body. As the ventricle relaxes, the piezoelectric induced power is released into the neighboring electrodes.

In another embodiment of the present invention, the power source of the respective exemplary embodiment outlined above can comprise at least one dopant, such as, for example and without limitation, a metallic agent such as cobalt, manganese, iron, copper, potassium, sodium, yttrium, titanium, lithium, and the like. One skilled in the art will appreciate that by doping the nanowires with at least one dopant a change to the conducting properties of the nanowires can be effected. One skilled in the art will also appreciate that the conducting properties of the material have a significant influence on the piezoelectric response of nanowires. In one aspect, doping changes the carrier concentration of the nanowire and enhances the piezoelectric response by modulating the dielectric constant. Since the carrier concentration of the material can be effectively decreased by the doping, i.e., by introducing impurities at lattice sites, the dielectric constant and the piezoelectric coefficient is increased. In another aspect, the dopant inclusion may improve the mechanical properties and create longer generator lifetimes by adding stiffness to the nanostructured array. In a further aspect, conventional electrochemistry or a core-shell approach techniques can be utilized to isotropically disperse dopants into the crystal lattice of the piezoelectric to affect desired changes in the conducting properties of the nanowires. The electrochemical approach can easily be applied to the exemplary synthetic technique described below using the necessary precursor of dopant and an applied potential to the solution.

The core-shell approach uses a serial process, first building a core of the piezoelectric then building a shell of metal ions at the surface. This technique can also be accomplished using the hydrothermal growth approach. By coating the nanowires with a thin conformal metal oxide shell, for example but not limited to titanium oxide (TiO₂), aluminum oxide (Al₂O₃) and the like, the piezoelectric potential may be tuned to higher responses. In another aspect, the thin oxide shell may add stiffness to the wires adding to the generator lifetimes. One skilled in the art will appreciate the misfit strain that develops between the adjoined layers. In this aspect, the conformal metal oxide coating can accommodate much larger strains than conventional piezoelectric nanostructures. The larger strains create larger piezoelectric responses by limiting the strain relaxation to the nanowire core and homogenizing the strain distribution along the axial direction.

As noted above, to prevent fracture from the electrode, the stiffness of the nanowires may be altered by coating the nanowires in a conformal metal oxide shell of alumina (Al₂O₃) or titania (TiO₂) made by atomic layer deposition (ALD). The core-shell structure has also been theoretically reported to increase the piezoelectric potential, where even larger amounts of energy could be generated. The oxide shell adds stiffness to the NWs by increasing the Young's modulus, which resists the fracture strain at the base between the substrate and NW.

In another embodiment of the present invention, the power source of the respective exemplary embodiment outlined above can comprise at least one surfactant, such as, for example and without limitation, a molecular surface coatings that is capable of combining with surface irregularities or vacancies present in the crystal nanowires such as stearic acid, perfluorotetradecanoic acid (CH₃, F) and the like. In one aspect, applying the surfactant contribute to the carrier density of the formed array of nanowires. Further, such a self assembled monolayer (SAM) changes the carrier concentration of the nanowire and enhances the piezoelectric response by modulating the dielectric constant. Molecular dipoles of SAMs change the energy barriers that develop between NWs and the contacts and enable the “tuning” of contact resistances to extract more energy from the NWs. Tuning the contact resistance with SAMs can be accomplished by placing the NW arrays and device into a bath of stearic acid for 12 hours and rinsing thoroughly with deionized water. Since the carrier concentration of the material can be effectively decreased by the doping, i.e., by introducing impurities at lattice sites, the dielectric constant and the piezoelectric coefficient is increased. In another aspect, the dopant inclusion may improve the mechanical properties and create longer generator lifetimes by adding stiffness to the nanostructured array.

Using the predicted load (˜40 μN) and the direct piezoelectric effect relationship, a single array of 10¹¹ NWs of the present invention would be able to produce at least 12 μW worth of power, compared to ˜0.5 μW, for conventional PVDF piezoelectric films. Thus, the estimated time to fully recharge an AICD and BVP battery would be approximately two years.

Fabrication of an Exemplary Piezoelectric Assembly

In an exemplary fabrication that is not meant to be limiting, polyimide (PI) substrates (25 μm thickness, Kapton HN, Dupont) were initially washed with acetone and isopropanol, rinsed with deionized water thoroughly and dried with a stream of nitrogen. The cleaned surfaces were then treated with a short Reactive Ion Etching (RIE, March Plasma CS1701F RIE etching system) oxygen plasma (20 sccm O₂ flow, 50 W, 10 seconds) to promote adhesion with the photoresist (AZ5209E, Positive Resist, Microchemicals). Gold (Au) electrode pads were then patterned on the PI substrates using a conventional liftoff technique. This exemplary substrate is not meant to limiting as poly ethyleneterephthalate (PET) substrates (100 μm thickness, Mylar, Grafix Plastics) and the like could also have been used, but PI substrates are used herein for clarity of the example. In one aspect, the piezoelectric assembly 50 can be grown on base electrodes, each of which is connected to a large interconnect that can be accessed externally conventionally. The exemplary piezoelectric assembly 50 also has a upper electrode that is connected to the NWs/PVDF films with a silver-based conducting epoxy.

In this exemplary non-limiting example, the preparation of the PVDF composite structures used a two-step process. First, PVDF-TrFE was dissolved in 2-butanone solvent overnight. Subsequently, the solvent was spin-coated onto the electrode pads and heated to 90 C for 30 seconds to ensure adhesion. Next, additional layers of PVDF were spin coated on top of the already cured layer, followed by another round of heating to ensure adhesion. In this fashion, a stack of PVDF films were deposited to form the built up composite structure. In a further aspect, it is contemplated that an inert material layer or film can be positioned between each layer of PVDF nanofilms forming the built up composite structure. By repeating this process, a stack of PVDF films were deposited to form the composite multilayer structure where the thickness of the individual layers could be anywhere from nanometers to many microns.

In an optional, exemplary non-limiting example the preparation of the PVDF composite structures composed of at least one PVDF layer used a two-step process. First, PVDF-TrFE was dissolved in 2-butanone solvent overnight. Subsequently, the PVDF-2-butanone solution was spin-coated onto the electrode pads and heated to 90 C for 30 seconds to ensure adhesion. Next, an additional layer, such as a secondary conducting or insulating polymer, was spin coated on top of the PVDF layer using a different solvent, followed by another round of heating to ensure adhesion. By repeating this process, a stack of PVDF films were deposited to form the composite multilayer structure in which the alternating layer structure substantially confines or isolates the spaced PVDF layers between intervening layers that are not formed from PVDF.

In a further aspect, multilayer PVDF-TrFE energy harvesting composite structures, such as those exemplary described above, having layers between about 1 to about 1000 nm thick can be made to maximize the energy harvesting capabilities of the formed composite structures.

To demonstrate that desired multilayer PVDF-TrFE energy harvesting composite structures can be made and that they do undergo crystallization, several preliminary experiments have been performed. In one aspect, using crosslinkable polystyrene (XST), multilayer films containing the desired nanoconfined PVDF-TrFE layers were formed according to the following process. First, PVDF-TrFE from cyclopentanone solution was spin coated onto a substrate and dried. The desired thickness confirmed using spectroscopic ellipsometry. Second, uncross-linked XST polymer from toluene solution was spin coated directly on top of previously formed PVDF-TrFE layer. As one skilled in the art will appreciate, PVDF-TrFE polymers are not soluble in nonpolar solvents. Next, the deposited XST film layer was cross-linked by conventional heat treatment at about 250° C. for about 5 minutes or by UV exposure at room temperature. As one skilled in the art will further appreciate, cross-linking of XST renders it insoluble in solvent and allows spin coating of subsequent PVDF-TrFE layers. Subsequently, the desired thickness of XST layer can be confirmed with spectroscopic ellipsometry. It is contemplated that the sequential application of the described steps can be repeated as desired to form a desired number of bilayer stacks in the multilayer PVDF-TrFE energy harvesting composite structures is achieved.

The process described above can be used to produce nanoconfined multilayer PVDF-TrFE films using XSTs or, instead of a spin coated insulating polymer, evaporated electrically conducting metals such as Ag, Au, Au/Pd. For example, and without limitation, two prototype samples were made with XSTs as a proof of principle; one prototype sample having 12 total layers, with an average PVDF-TrFE layer thickness of 99 nm and an average XST layer thickness of 54 nm, and a second prototype sample having 20 total layers, with an average PVDF-TrFE layer thickness of 64 nm and an average XST layer thickness of 40 nm. The standard deviation of the layer thickness in the stack was less than 10 nm. Using differential scanning calorimetry (DSC), the crystallization behavior of these samples was characterized and the data provided clear evidence that crystallization processes are impacted by confinement of PVDF-TrFE into nanolayers (both peak locations and shapes change appreciably). The described multilayer PVDF-TrFE energy harvesting composite structures exploit the fact that crystallization processes are enhanced in such a way that piezoelectric response is improved over thicker films, i.e., films having a layer thickness greater than about 100 nm).

The preparation of the oriented piezoelectric NW arrays composed of ZnO used a two-step process. In this example, a synthetic approach was used to grow oriented piezoelectric nanowires on plastic substrates that can be interfaced with AICD/BVP leads. First, using a deposition mask, crystallites of the piezoelectric material were spin-casted onto the electrode pads and heated to 100° C. for 30 seconds to ensure adhesion. Next, textured nanoplatelets were grown directly on the base electrode by tempering to 200° C. for twenty minutes. The piezoelectric assembly 50 is grown from the textured nanoplatelets using a growth procedure described below. In this fashion, NW arrays were grown hydrothermally from each type of ZnO seed at 92° C. in aqueous solution of 0.025M zinc nitrate hexahydrate (Zn(NO₃)₂.6H₂O), 0.025M hexamethylenetetramine (C₆H₁₂N₄) and 0.007M branched low-molecular weight polyethylenimine (PEI) for 36 hours. The arrays were then rinsed thoroughly with deionized water and baked at 80° C. overnight to remove any residual organics. TEM characterization of individual NWs removed from the arrays indicates that they are single-crystalline ZnO and grow substantially normal to the surface.

The respective NW arrays were grown from catalyst seeds. In one exemplary aspect, textured nanoplatelets were used in order to improve the orientation of the seed layer. In this aspect, the textured nanoplatelets had their c-axis textured to lie substantially perpendicular to the surface while maintaining the high surface to volume ratio of the nanoplatelet. FIG. 21 shows a representative SEM image of NWs grown from the textured nanoplatelets. The resulting nanowire array is extremely dense (10¹⁰ wires/cm²) with epitaxial orientation. The orientation was quantified and shows a high degree of alignment.

Further, in order to anchor the nanowires to the contact pads and prevent potential short circuits due to pinholes in the NW array when the upper electrode is introduced, a polymer layer was grafted onto the NWs to secure the NWs to the bottom contact electrodes and to provide mechanical stability to the array. In this exemplary fabrication, an adhesion promoter (AP150, Silicon Resources Inc.) was first dropped onto the NWs and is heated to 85° C. for 1 minute. The molecular layer of AP150 chemically bonds the NWs to the surrounding polymer. Next, a solution of monomer (Methyl Methacrylate, Sigma-Aldrich) and photoinitiator (Irgacure 651, Ciba) was dropped onto the array and spun at 3000 rpm for 30 seconds (Spincoater, Laurell Technologies). The array was subsequently degassed to remove any trapped air and photopolymerized using ultraviolet light. The NWs and polymer were then etched with an Ar—O₂ plasma (10 sccm Ar flow, 30 sccm O₂ flow, 50 W, 30 seconds) to expose the tops of the wires (FIG. 10). The length of the wires protruding from the polymer was controlled by the plasma etching time. As previously discussed, it is also contemplated that the anchoring layer could also be PVDF, a piezoelectric polymer, PVDF with other compounds of TrFE and BaTiO₃, and the like.

Subsequently, a flexible silver-based conducting epoxy was cast over the NW tips to provide the upper electrode. A liquid polyimide (PI-2770, HD Microsystems) was then cast over the device, developed with UV light, and post-cured at 100° C. for six minutes. The PI layer enables another device to be processed on top for potential three-dimensional architectures. As one would appreciate, the wires are good conductors along the direction of the wire axes and form excellent electrical junctions with the neighboring contacts. Two-point electrical measurements of the devices gave linear current-voltage (I-V) traces, indicating low contact resistance between NWs and contacts.

In an optional aspect and referring now to FIGS. 22-27, an exemplary piezoelectric assembly having a piezoelectric generator using PVDF nanowires or nanofilms was demonstrated. In this example, a polymer solution containing 80% PVDF dissolved in trifluoroethanol (TFE) was electrospun to mechanically stretch and electrically pole the PVDF nanowires to align the β-phase of all of the respective PVDF nanowires. The as-spun PVDF nanowires can exemplary be, without limitation about 200-300 nm in diameter and 2-3 cm in length. For testing purposes, and as shown in FIG. 22, a single PVDF nanowire or nanofilm was positioned on the underlying surface of a Kapton polyimide film substrate with the nanowire's or nanofilm's respective ends being bonded to the substrate using silver paste.

As shown in FIGS. 24 to 27, when the formed Kapton polyimide film substrate was bent via the application of external force, a pure axial strain was experienced by the PVDF nanofilm and nanowire and a piezoelectric potential was generated. FIG. 26 shows the voltage outputs of the experimental set up shown in FIG. 22, when the respective positive and negative probes of the measurement system were coupled to the respective positive and negative ends of the PVDF-TrFE nanofilm. As shown in FIG. 26, the positive peak A corresponds to the stretching state of the PVDF-TrFE nanofilm when the underlying substrate was mechanically bent. As the strain was released from the PVDF-TrFE nanofilm, the corresponding negative peak B is observed in the output voltage measurement. To find the correlation of the strain with the voltage output from the devices, two parallel cantilever beams were suspended form a rigid end. One of the cantilever beam carried the PVDF devices and the other cantilever beam carried the stain gage sensor. The free end of the cantilever beam was flexed using a displacement stage.

To test whether the received signal was showing the true electricity output due to the piezoelectric property of the PVDF-TrFE nanofilm and was not an artifact of the measurement system, the polarity of the probes was reversed and strain was again subsequently applied and released to the substrate. Thus, in the reverse connection configuration, the respective positive and negative probes of the measurement system were coupled to the respective negative and positive ends of the PVDF-TrFE nanofilm. As shown in FIG. 27, the stretching of the PVDF-TrFE nanofilmas the substrate was mechanically bent produced a negative peak a and a positive peak b when released. This result demonstrates that the received signal was a result of the piezoelectric potential and was not signaling a capacitor change.

The tests further demonstrated that the use of PVDF nanowires/nanofilms have the potential for generating significantly higher voltage outputs then comparative nanowires formed from ZnO. As shown in FIG. 24, an exemplary PVDF nanowire generated a voltage output of approximately 15 mV while similar comparative experiments with ZnO nanowires show a generated voltage output that is typically less than 2 mV.

Although several aspects of the present invention have been disclosed in the foregoing specification, it is understood by those skilled in the art that many modifications and other aspects of the invention will come to mind to which the invention pertains, having the benefit of the teaching presented in the foregoing description and associated drawings. It is thus understood that the invention is not limited to the specific aspects disclosed hereinabove, and that many modifications and other aspects are intended to be included within the scope of the appended claims. Moreover, although specific terms are employed herein, as well as in the claims which follow, they are used only in a generic and descriptive sense, and not for the purposes of limiting the described invention.

1. An implant configured for implantation inside a human body, comprising:
a power consuming means for responding to a physiological requirement of the body; and
a power source comprising a flexible piezoelectric assembly configured to generate an electrical current when flexed by a tissue in the human body, wherein the piezoelectric assembly comprises a plurality of oriented piezoelectric polymer nanowires arranged in an array and forming a nanowire layer or a plurality of piezoelectric polymer nanofilm layers forming a piezoelectric composite structure.

2. The implant of claim 1, wherein the piezoelectric polymer nanowires comprises at least one polyvinylidene fluoride (PVDF) nanowire.

3. The implant of claim 2, wherein the piezoelectric poloymer nanofilm layers comprises at least one PVDF nanofilm layer.

4. The implant of claim 1, wherein the piezoelectric assembly is sheathed, and wherein the plurality of oriented nanowires is encapsulated in a polymeric matrix.

5. The implant of claim 1, wherein the piezoelectric assembly comprises a lower electrode, and wherein the plurality of nanowires extend outwardly from the lower electrode and are oriented generally parallel to a common array axis relative to the lower electrode.

6. The implant of claim 5, wherein the common array axis is oriented at between about 70° to 110° relative to the lower electrode.

7. The implant of claim 5, wherein the common array axis is oriented at about 90° relative to the lower electrode.

8. The implant of claim 1, further comprising a power storage device electrically coupled to the power source and to the power consuming means.

9. The implant of claim 5, wherein the piezoelectric assembly comprises an upper electrode layer that opposes the lower electrode layer, and wherein the plurality of nanowires are operatively coupled to the upper electrode layer and the opposed lower electrode layer.

10. The implant of claim 1, wherein the plurality of nanowires is formed from an array of piezoelectric crystals.

11. The implant of claim 10, wherein the piezoelectric crystals comprise PVDF crystals.

12. The implant of claim 1, wherein the piezoelectric assembly comprises a plurality of nanowire layers that are positioned in stacked relationship.

13. The implant of claim 9, wherein the piezoelectric assembly further comprises at least one sheet of flexible piezoelectric film.

14. The implant of claim 9, wherein the sheathed piezoelectric assembly comprises a plurality of layers selected from a group consisting of at least one nanowire layer and at least one sheet of flexible piezoelectric film.

15. The implant of claim 1, wherein the plurality of piezoelectric polymer nanofilm layers are positioned in stacked relationship.

16. The implant of claim 15, wherein the plurality of piezoelectric polymer nanofilm layers further comprises at least one sheet of flexible PVDF nanofilm.

17. The implant of claim 15, wherein the piezoelectric composite structure further comprises a plurality of secondary layers interposed between respective piezoelectric polymer nanofilm layers.

18. The implant of claim 17, wherein the piezoelectric poloymer nanofilm layers comprises at least one PVDF nanofilm layer, and wherein the plurality of secondary layers is not formed of PVDF.

19. The implant of claim 1, wherein the power consuming means comprises an AICD, BiVentricular Pacemaker, or other implantable power consuming devices placed within the human body.

20. The implant of claim 19, wherein the AICD comprises a pacing lead having a proximal electrode and a spaced distal electrode, wherein the power source is encapsulated within an intermediate portion of the pacing lead between the respective proximal and distal electrodes.

21. The implant of claim 20, wherein the power source is arranged in a spiral configuration within the intermediate portion of the pacing lead.

22. The implant of claim 20, wherein the power source is mounted to an interior surface of a wall of the pacing lead.

23. The implant of claim 1, wherein the power consuming means comprises a BVP.

24. The implant of claim 23, wherein the BVP comprises a pacer lead that is positioned along the left ventricular outer wall, and wherein the power source is encapsulated within a portion of the pacer lead.

25. The implant of claim 1, wherein the each nanowire or nanofilm comprises at least one dopant.

26. The implant of claim 1, wherein at least a portion of each nanowire or nanofilm is coated in a conformal metal oxide shell.

27. The implant of claim 1, wherein at least a portion of each nanowire or nanofilm is treated with a surfactant.

28. The implant of claim 27, wherein the surfactant comprises a self-assembled monolayer.

29. The implant of claim 9, wherein at least a portion of the electrodes are treated with a molecular surface coating.

30. The implant of claim 29, wherein the molecular surface coating comprises a self-assembled monolayer.

31. The implant of claim 1, wherein the power consuming means comprises at least one of an AICD, a BVP, a pacemaker, monitoring systems, pressure and volume detectors to warn of impending heart failure, piggybacked chemical sensors for diabetics to measure glucose, potassium, and renal function (BUN and creatinine), artificial hearts, and left and right ventricular assist devices.

32. The implant of claim 9, wherein the respective upper and lower electrodes are formed into periodic wave-like geometries.

33. An implant configured for implantation inside a human body, comprising:
a power source comprising a flexible piezoelectric assembly configured to generate an electrical current when flexed by the tissue of the body, wherein the piezoelectric assembly comprises an upper electrode, an opposed lower electrode, and a plurality of nanowires arranged in an array and forming a nanowire layer, wherein the plurality of nanowires extend upwardly from a lower electrode layer, wherein each of the plurality of nanowires is generally oriented parallel to a common array axis, and wherein the plurality of oriented nanowires comprises a plurality of polyvinylidene fluoride (PVDF) nanowires.

34. The implant of claim 33, further comprising:
a power consuming means for responding to a physiological requirement of the body; and
a power storage device electrically coupled to the power source and to the power consuming means.

35. The implant of claim 33, wherein the plurality of nanowires is formed from an array of piezoelectric crystals.

36. The implant of claim 34, wherein the power consuming means comprises an AICD.

37. The implant of claim 34, wherein the power consuming means comprises a BVP.

37. A method of measuring the ventricular function of a heart, comprising;
providing an implant comprising:
a power consuming means for responding to a physiological requirement of the body;
a power source comprising a flexible piezoelectric assembly configured to generate an electrical current when flexed by the tissue of the body, wherein the piezoelectric assembly comprises an upper electrode, an opposed lower electrode, and a plurality of nanowires arranged in an array and forming a nanowire layer, wherein each of the plurality of nanowires is generally oriented parallel to a common array axis, wherein the plurality of nanowires are operatively coupled to the upper electrode layer and the opposed lower electrode layer, and wherein the plurality of oriented nanowires comprises a plurality of polyvinylidene fluoride (PVDF) nanowires; and
a power storage device electrically coupled to the power source and to the power consuming means;
measuring the current generated by the power source; and
calculating the strength of the heart's contraction from the measured current generated by power source.
